(19) United States
(12) Patent Application Publication
(10) Pub. No.: US 2017/0150800 A1
(43) Pub. Date: Jun. 1, 2017

(54) STABILIZED HYDROGEN PEROXIDE FORMULATIONS IN SACHETS MADE OF BARRIER LAYER FILMS

(71) Applicant: Henkel AG & Co. KGaA, Duesseldorf (DE)

(72) Inventors: Burkhard Mueller, Duesseldorf (DE); Norbert Schettiger, Hilden (DE); Marc Krippahl, Moenchengladbach (DE); Wolfgang Barthel, Langenfeld (DE)

(73) Assignee: Henkel AG & Co. KGaA, Duesseldorf (DE)

(21) Appl. No.: 15/354,015

(22) Filed: Nov. 17, 2016

(30) Foreign Application Priority Data
Dec. 1, 2015 (DE) 10 2015 223 838.5

(51) Int. Cl. A45D 37/00; A61K 8/22; A61K 8/24; A61K 8/55; A61K 8/368; A61Q 5/06; A61K 8/49

(52) U.S. Cl. CPC A45D 37/00; A61Q 5/065; A61K 8/22; A61K 8/4926; A61K 8/55; A61K 8/368; A61K 8/24; A61K 2800/52; A61K 2800/87; A61K 2800/4322

(57) ABSTRACT

A cosmetic product for the oxidative color changing of keratinic fibers, in particular human hair, comprising
a preparation (A), which is packaged in a sachet (S), wherein
preparation (A) includes
   (A1) water and
   (A2) hydrogen peroxide and
   (A3) at least one stabilizer from the group comprising 2,6-dipicolinic acid, 1-hydroxyethane-1,1-diphosphonic acid (HEDP), benzoic acid, salicylic acid, ethylenediamine tetra(methylenephosphonic acid) (EDTMP), diethylenetriamine penta(methylenephosphonic acid) (DTPMP), amino tris(methylenephosphonic acid) (ATMP), N,N-bis[2-[bis(carboxymethyl)-amino]ethyl]glycine, ethylenediamine-N,N′-disuccinic acid (EDDS), 2-hydroxypropylenediamine-N,N′-disuccinic acid (HPDDS), ethylenediamine-N,N′-diglutaric acid (EDDG), ethylenediamine-N,N′-bis(orthohydroxyphenyl)acetic acid (EDDHA), diphosphoric acid, hydrated tin oxide,
      and/or the physiologically acceptable salts thereof,
the wall of sachet (S) comprises
   (S1) a first layer made of a first polymeric material and
   (S2) a second layer made of a second polymeric material and
   (SBa) a barrier layer, which has a penetration barrier effect for gases and water vapor.

### FIELD OF THE INVENTION

The present invention generally relates to cosmetics, and more particularly relates to a product for the oxidative color changing of keratinic fibers, in particular human hair, which comprises an aqueous hydrogen peroxide preparation packaged in a sachet. The hydrogen peroxide preparation is characterized by the content of specific stabilizers. The sachet, which is used as the packaging for the hydrogen peroxide preparation, is a sachet which is fabricated from a special multilayer film composite system and whose wall comprises at least two polymer layers and one barrier layer. In this case, the barrier layer has a penetration barrier effect for gases and water vapor.

### BACKGROUND OF THE INVENTION

The changing of the color of keratinic fibers, particularly of hair, is an important field in modern cosmetics. Because of this, the appearance of the hair can be adapted both to current fashion trends and to the individual wishes of the individual person. The skilled artisan is aware of various options for changing the color of hair.

Hair color can be changed temporarily by the use of direct dyes. In this case, already formed dyes diffuse from the coloring agent into the hair fiber. Dyeing with direct dyes is associated with less hair damage, but a disadvantage is the low durability and the rapid washing out of the colors obtained with direct dyes.

Oxidative color-changing agents are usually used therefore if the consumer wants a long-lasting color result or a shade that is lighter than the consumer's original hair color. So-called oxidation dyes are used for permanent, intensive colors with suitable fastness properties. Such coloring agents typically include oxidation dye precursors, so-called developer components and coupler components, which together form the actual dyes under the influence of oxidizing agents, usually hydrogen peroxide. Oxidation dyes are characterized by excellent, long-lasting color results.

The mere lightening or bleaching of hair often occurs with the use of oxidizing agents without the addition of oxidation dye precursors. The use of hydrogen peroxide alone as an oxidizing agent is sufficient for an average bleaching effect; a mixture of hydrogen peroxide and peroxydisulfate salts is generally employed to achieve a greater bleaching effect.

Oxidative color-changing agents are typically found on the market in the form of two-component agents, in which the two different preparations are packaged separately in two separate packages and are mixed together only shortly before use.

The first preparation is a formulation, which has been made acidic for reasons of stability and which includes hydrogen peroxide in concentrations of 1.5 to 12% by weight as the oxidizing agent. The oxidizing agent formulation mostly has the form of an emulsion or dispersion and as a rule is made available in a plastic bottle with a reclosable outlet opening (developer bottle).

This hydrogen peroxide formulation is mixed before use with a second preparation. This second preparation is a formulation, which has been made alkaline and is often available in the form of a cream or a gel and which, provided that a change in color is desired concurrently with the lightening, also includes in addition oxidation dye precursors. This second preparation can be provided, for example, in the form of a tube or in the form of a plastic or glass container.

In the case of the previously described most customary application form, the second preparation, which includes the alkalizing agent and/or the oxidation dye precursors, is transferred from the tube or container to the developer bottle and then mixed by shaking with the hydrogen peroxide preparation already present in the developer bottle. The application mixture is prepared in the developer bottle in this way. The application to the hair then occurs via a small spout or outlet opening at the top of the developer bottle. The spout or outlet opening is opened after the shaking, and the application mixture can be removed by pressing the flexible developer bottle.

The use of the developer bottle requires a certain routine from the user, so that some users prefer preparing the application mixture in a mixing bowl and applying it using a brush.

When the application mixture is prepared in a bowl, both components, that is, the first preparation including the hydrogen peroxide and the second preparation with the alkalizing agent and/or oxidation dye precursors, are transferred completely to a bowl or a similar vessel and there stirred, for example, with the aid of a brush. The application mixture is then removed from the mixing bowl with use of the brush. In this application form, the use of a voluminous and expensive developer bottle is not necessary, and the search continues for inexpensive and material-saving packaging forms for the preparation with hydrogen peroxide.

The sachet lends itself in this regard as an inexpensive packaging form with a low material consumption. A sachet is a small package in a bag or pouch form, which is often used in the packaging of cosmetics. Sachets are generally made of plastic films or also metal foils.

A typical sachet can be produced, for example, by gluing or hot pressing two plastic films lying one above the other, the gluing occurring on all edges of the films. The interior of the sachet (i.e., of the plastic bag) produced by gluing can then be filled with the desired cosmetic preparation. The sachet can be opened by tearing open or cutting of the plastic bag.

The filling of hydrogen peroxide preparations into sachets is associated with problems, however, whose cause resides in the reactivity of the peroxide. Hydrogen peroxide is a highly reactive substance, which, depending on the storage conditions and optionally on the presence of contaminants with a decomposing action, breaks down in small amounts with the formation of oxygen (i.e., of gas).

The developer bottles known from the prior art are generally filled with the hydrogen peroxide formulation at most only to half, typically only to a third of their inner volume. Developer bottles are generally made of polyethylene. Because polyethylene is permeable to both water vapor and gases, no or only a very low excess pressure forms in the developer bottle. Moreover, developer bottles are typically provided with stable, thick walls and a stable screw closure, so that the diffusion of the water vapor or gases is reduced by the thickness of the walls and an increase in pressure occurring to a limited extent in the bottle has only little influence.

In contrast, however, sachets are usually completely filled with the liquid preparation, and there is virtually no remaining air space in the filled sachet. In addition, a sachet is to be flexible, and when the sachet is opened (e.g., torn or cut open), there should be no uncontrolled escape of the preparation. For this reason, during packaging of liquid preparations, the formation of excess pressure in the sachet should be avoided if possible.

If there is a hydrogen peroxide preparation in the sachet, thus the gas (oxygen) forming during storage can lead to swelling of the sachet. Because the edges of the sachet typically are only glued, in the worst case great swelling leads to bursting of the sachet. For these reasons, when hydrogen peroxide-containing preparations are packaged, the choice of the film material comprising the sachet is of the greatest importance.

Sachets, which consist of pure plastic such as, for example, polyethylene or polypropylene, are permeable to both water vapor and gases. When a hydrogen peroxide-containing preparation is packaged in a sachet made of polyethylene or polypropylene, no swelling of the sachet occurs for this reason. Because of the high permeability of the relatively thin film of the sachet to water vapor, the water content of the preparation decreases, however. If the preparation in the sachet is stored for a few weeks to months, the water loss exceeds any acceptable level.

Completely air-tight sachets are produced, for example, from plastic films, which have a lamination with a metal layer, for example, with an aluminum layer. These sachets are completely impermeable to water vapor and gases. If these sachets are filled with a hydrogen peroxide-containing preparation, thus the gas forming during the decomposition of the peroxide cannot escape, and the sachet swells as previously described and bursts.

The object of the present invention was to package the hydrogen peroxide preparation inexpensively, with the saving of material and space, securely, and in particular with stability during storage in the form of a sachet.

Surprisingly, it has now emerged that hydrogen peroxide preparations can be packaged in a storage stable manner in the sachet, if sachets are used for packaging, which sachets consist of special film composite systems and have a barrier layer, and if these sachets are filled with hydrogen peroxide preparations, containing special stabilizers.

Furthermore, other desirable features and characteristics of the present invention will become apparent from the subsequent detailed description of the invention and the appended claims, taken in conjunction with this background of the invention.

### BRIEF SUMMARY OF THE INVENTION

A cosmetic product for the oxidative color changing of keratinic fibers, in particular human hair, comprising a preparation (A), which is packaged in a sachet (S), wherein preparation (A) includes water and hydrogen peroxide and at least one stabilizer from the group comprising 2,6-dipicolinic acid, 1-hydroxyethane-1,1-diphosphonic acid (HEDP), benzoic acid, salicylic acid, ethylenediaminetetraacetic acid (EDTA), ethylenediamine tetra(methylenephosphonic acid) (EDTMP), diethylenetriamine penta(methylenephosphonic acid) (DTPMP), amino tris(methylenephosphonic acid) (ATMP), N,N-bis[2-[bis(carboxymethyl)amino]ethyl]glycine, ethylenediamine-N,N′-disuccinic acid (EDDS), 2-hydroxypropylenediamine-N,N′-disuccinic acid (HPDDS), ethylenediamine-N,N′-diglutaric acid (EDDG), ethylenediamine-N,N′-bis(orthohydroxyphenyl)acetic acid (EDDHA), diphosphoric acid, hydrated tin oxide; and/or the physiologically acceptable salts thereof, and the wall of sachet (S) comprises: a first layer made of a first polymeric material, a second layer made of a second polymeric material, and a barrier layer, which has a penetration barrier effect for gases and water vapor.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

The subject of the present invention is a cosmetic product for the oxidative color changing of keratinic fibers, in particular human hair, comprising
a preparation (A), which is packaged in a sachet (S), wherein
preparation (A) includes
   (A1) water and
   (A2) hydrogen peroxide and
   (A3) at least one stabilizer from the group comprising 2,6-dipicolinic acid, 1-hydroxyethane-1,1-diphosphonic acid (HEDP), benzoic acid, salicylic acid, ethylenediaminetetraacetic acid (EDTA), ethylenediamine tetra(methylenephosphonic acid) (EDTMP), diethylenetriamine penta(methylenephosphonic acid) (DTPMP), amino tris(methylenephosphonic acid) (ATMP), N,N-bis[2-[bis(carboxymethyl)-amino]ethyl]glycine, ethylenediamine-N,N′-disuccinic acid (EDDS), 2-hydroxypropylenediamine-N,N′-disuccinic acid (HPDDS), ethylenediamine-N,N′-diglutaric acid (EDDG), ethylenediamine-N,N′-bis(orthohydroxyphenyl)acetic acid (EDDHA), diphosphoric acid, hydrated tin oxide, and/or the physiologically acceptable salts thereof, and
the wall of sachet (S) comprises:
   (S1) a first layer made of a first polymeric material and
   (S2) a second layer made of a second polymeric material and
   (SBa) a barrier layer, which has a penetration barrier effect for gases and water vapor.

Keratinic fibers, keratin-containing fibers, or keratin fibers are to be understood to mean pelts, wool, feathers, and in particular human hair. Although the agents of the invention are primarily suitable for lightening and coloring keratin fibers, in principle nothing precludes use in other fields as well.

The product of the invention is a product for the oxidative color changing of keratinic fibers, i.e., a product that is used on the human head so as to achieve an oxidative dyeing, lightening, blonding, bleaching, or nuancing of the hair. Nuancing in this regard is understood to mean a dyeing in which the color result is lighter than the original hair color.

The product of the invention comprises a preparation (A), which is packaged in a sachet (S) and is characterized by its content of water (A1) and hydrogen peroxide (A2) and one or more stabilizers from group (A3).

The intended application of the product of the invention is oxidative color changing. To this end, as already previously described, a preparation (A), which includes hydrogen peroxide, is typically mixed with a second preparation (B), packaged separately from (A). The ready-to-use oxidative color-changing agent is produced in this way. Depending on whether a blonding, lightening, or dyeing is to be achieved with the oxidative color changing, preparation (B) may include different ingredients. If just a lightening or blonding is to be achieved, preparation (B) includes at least one alkalizing agent. If an oxidative dyeing is desired, thus preparation (B) also often includes oxidation dye precursors apart from the alkalizing agent. In order to assure a sufficiently rapid mixability of preparations (A) and (B), both preparation (A) and preparation (B) are typically flowable, aqueous, or water-containing preparations.

Preparation (A) according to the invention is an aqueous preparation. The water content of preparation (A), based on the total weight of preparation (A), can be, for example, 60.0 to 97.0% by weight, preferably 75.0 to 93% by weight, more preferably 78.0 to 91% by weight, and particularly preferably 80.0 to 88.0% by weight. All weight data given in % by weight in this case relate to the amount by weight of water (A1), which is included in preparation (A) and is related to the total weight of preparation (A).

In a particularly preferred embodiment, a product of the invention is characterized in that preparation (A) includes, based on the total weight of preparation (A), water (A1) in an amount of 60.0 to 97.0% by weight, preferably 75.0 to 93% by weight, more preferably 78.0 to 91% by weight, and particularly preferably 80.0 to 88.0% by weight.

Furthermore, preparation (A) includes hydrogen peroxide as the second feature (A2), essential to the invention. The concentration of hydrogen peroxide in preparation (A) is determined, on the one hand, by legal requirements and, on the other, by the desired effect; preferably, 0.5 to 20.0% by weight solutions in water are used. Preparations (A) preferred according to the invention are characterized in that, based on the total weight of preparation (A), they include hydrogen peroxide (A2) in an amount of 0.5 to 20.0% by weight, preferably 1.5 to 17.0% by weight, more preferably of 1.5 to 15.0% by weight, and particularly preferably of 1.5 to 12.0% by weight.

The higher the content of hydrogen peroxide (A2) in preparation (A), the higher the amount of gas forming in a partial decomposition of hydrogen peroxide. More highly concentrated hydrogen peroxide preparations accordingly are much more difficult to package in a sachet in a storage stable manner than less concentrated preparations.

During the work leading to this invention, it emerged that the product of the invention is also particularly suitable for the packaging and stable storage of more highly concentrated hydrogen peroxide preparations (A). Thus, sachets (S) of the invention, which included preparations (A) with 9 to 12% by weight of hydrogen peroxide (A2), exhibited no changes in volume (i.e., no swelling) even after weeks of storage at an increased temperature and no unplanned openings (i.e., the sachets did not burst).

For this reason, the cosmetic products are very particularly preferred in which preparation (A), packaged in sachet (S), includes hydrogen peroxide (A2) in an amount of 1.5 to 12.0% by weight, preferably 3.0 to 12.0% by weight, more preferably 6.0 to 12.0% by weight, and very particularly preferably 9.0 to 12.0% by weight. All weight data given in % by weight in this case relate to the amount by weight of hydrogen peroxide (A2), which is included in preparation (A) and is related to the total weight of preparation (A).

In another particularly preferred embodiment, a product of the invention is characterized in that preparation (A) includes, based on the total weight of preparation (A), hydrogen peroxide (A2) in an amount of 1.5 to 12.0% by weight, preferably 3.0 to 12.0% by weight, more preferably 6.0 to 12.0% by weight, and very particularly preferably 9.0 to 12.0% by weight.

Preparation (A) includes as the third ingredient, essential to the invention, one or more stabilizers (A3), which are selected from the group comprising 2,6-dipicolinic acid, 1-hydroxyethane-1,1-diphosphonic acid (HEDP), benzoic acid, salicylic acid, ethylenediaminetetraacetic acid (EDTA), ethylenediamine tetra(methylene phosphonic acid) (EDTMP), diethylenetriamine penta(methylene phosphonic acid) (DTPMP), amino tris(methylene phosphonic acid) (ATMP), N,N-bis[2-[bis(carboxymethyl)amino]ethyl]glycine, ethylenediamine-N,N′-disuccinic acid (EDDS), 2-hydroxypropylenediamine-N,N′-disuccinic acid (HPDDS), ethylenediamine-N,N′-diglutaric acid (EDDG), ethylenediamine-N,N′-bis(orthohydroxyphenyl)acetic acid (EDDHA), diphosphoric acid, hydrated tin oxide, and/or the physiologically acceptable salts of these compounds.

Stabilizers in the context of the present invention are substances that are capable of forming chelates or complexes that function as free radical scavengers and/or can form insoluble metal salts. It emerged that the aforementioned stabilizers from group (A3) are capable of stabilizing hydrogen peroxide and prevent the decomposition of hydrogen peroxide catalyzed by the heavy metals.

The physiologically acceptable salt(s) of the aforementioned stabilizers is/are understood to be the salts of the compounds which can be applied to the skin without disadvantageous toxicological effects. Physiologically acceptable salts are understood typically in particular to be the sodium salts, potassium salts, and the ammonium salts (NH₄⁺) of the aforementioned stabilizers.

2,6-Dipicolinic acid is also called alternatively 2,6-pyridinedicarboxylic acid; it is a compound with the formula (I) and has the CAS no. 499-83-2.

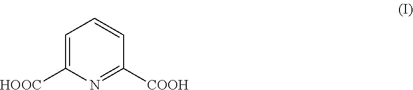 [SMILES: O=C(O)c1cccc(C(=O)O)n1]

Suitable physiologically acceptable salts of dipicolinic acid are, for example, the mono- or disodium salt or the mono- or dipotassium salt.

Benzoic acid is also called alternatively benzenecarboxylic acid and has the structure of the formula (II). Benzoic acid has the CAS no. 65-85-0.

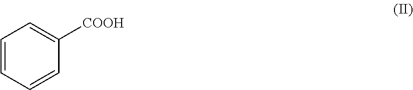

Suitable physiologically acceptable salts of benzoic acid are, for example, the sodium salt, potassium salt, or the ammonium salt (NH₄⁺).

Salicylic acid is also called alternatively 2-hydroxybenzoic acid and has the structure of the formula (III). Salicylic acid has the CAS no. 69-72-7.

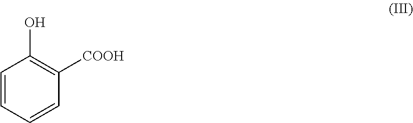

Suitable physiologically acceptable salts of benzoic acid are, for example, the sodium salt, potassium salt, or the ammonium salt (NH₄⁺).

1-Hydroxyethane-1,1-diphosphonic acid (HEDP) is also called alternatively etidronic acid; it is a compound with the formula (IV) and has the CAS number 2809-21-4.

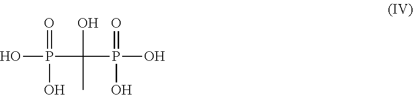

Suitable physiologically acceptable salts of HEDP are, for example, the mono-, di-, tri-, or tetrasodium salt, or the mono-, di-, tri-, or tetrapotassium salt.

Ethylenediaminetetraacetic acid (EDTA) is a compound of the formula (V); the substance has the CAS no. 60-00-4.

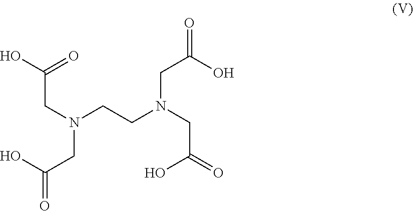 [SMILES: O=C(O)CN(CCN(CC(=O)O)CC(=O)O)CC(=O)O]

Suitable physiologically acceptable salts of EDTA are, for example, the mono-, di-, tri-, or tetrasodium salt, or the mono-, di-, tri-, or tetrapotassium salt.

Ethylenediamine tetra(methylene phosphonic acid) (EDTMP) is a compound of the formula (VI); the substance has the CAS no. 1429-50-1.

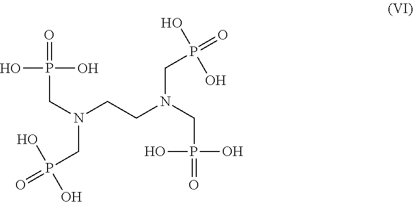 [SMILES: O=P(O)(O)CN(CCN(CP(=O)(O)O)CP(=O)(O)O)CP(=O)(O)O]

Suitable physiologically acceptable salts of EDTMP are, for example, the mono-, di-, tri-, or tetrasodium salt, or the mono-, di-, tri-, or tetrapotassium salt.

Diethylenetriamine penta(methylene phosphonic acid) (DTPMP) is a compound of the formula (VII); the substance has the CAS no. 15827-60-8.

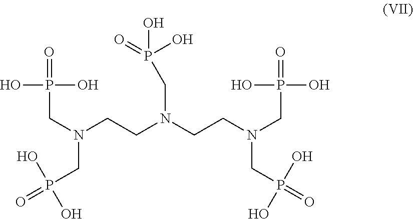 [SMILES: O=P(O)(O)CN(CCN(CP(=O)(O)O)CP(=O)(O)O)CCN(CP(=O)(O)O)CP(=O)(O)O]

Suitable physiologically acceptable salts of DTPMP are the mono-, di, tri-, tetra-, and pentasodium salt of this compound, and the mono-, di, tri-, tetra-, and pentapotassium salt of this compound.

Amino tris(methylene phosphonic acid) (ATMP) is also called alternatively nitrilotris(methylene phosphonic acid). ATMP has the formula (VIII) and has the CAS no. 6419-19-8.

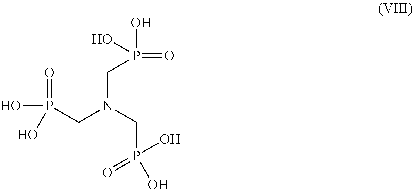 [SMILES: O=P(O)(O)CN(CP(=O)(O)O)CP(=O)(O)O]

Physiologically acceptable salts thereof are, for example, the mono-, di-, or trisodium salt of ATMP, or the mono-, di-, or tripotassium salt of this compound.

N,N-bis[2-[bis(carboxymethyl)amino]ethyl]glycine is a compound of the formula (IX). Alternative names for this compound are diethylenetriaminepentaacetic acid (DTPA) or also 1,1,4,7,7-diethylenetriaminepentaacetic acid. The compound has the CAS no. 67-43-6.

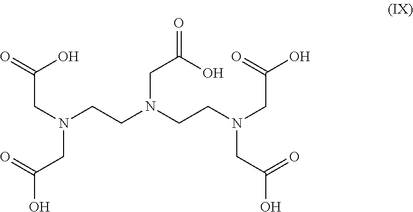 [SMILES: O=C(O)CN(CCN(CC(=O)O)CC(=O)O)CCN(CC(=O)O)CC(=O)O]

Suitable physiologically acceptable salts thereof are, for example, the mono-, di-, tri-, tetra-, or pentasodium salt or the mono-, di-, tri-, tetra-, or pentapotassium salt of this compound.

Ethylenediamine-N,N′-disuccinic acid (EDDS), alternatively also called ethylenediamine disuccinate, is a compound of the formula (X). The compound has the CAS no. 20846-91-7.

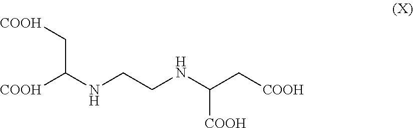 [SMILES: O=C(O)CC(NCCNC(CC(=O)O)C(=O)O)C(=O)O]

A suitable physiologically acceptable salt is, for example, the mono-, di-, tri-, or tetrasodium salt of the compound, or the mono-, di-, tri-, or tetrapotassium salt of this compound.

2-Hydroxypropylenediamine-N,N′-disuccinic acid (HPDDS) is a compound of the formula (XI).

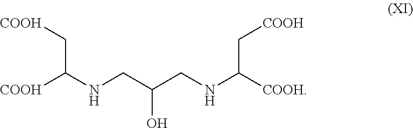 [SMILES: O=C(O)CC(NCC(O)CNC(CC(=O)O)C(=O)O)C(=O)O]

A suitable physiologically acceptable salt is, for example, the mono-, di-, tri-, or tetrasodium salt of the compound, or the mono-, di-, tri-, or tetrapotassium salt of this compound.

Ethylenediamine-N,N′-diglutaric acid (EDDG) is a compound of the formula (XII).

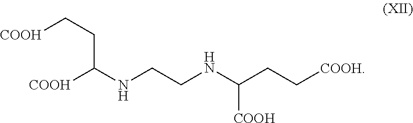 [SMILES: O=C(O)CCC(NCCNC(CCC(=O)O)C(=O)O)C(=O)O]

A suitable physiologically acceptable salt is, for example, the mono-, di-, tri-, or tetrasodium salt of the compound, or the mono-, di-, tri-, or tetrapotassium salt of this compound.

Ethylenediamine-N,N′-bis(orthohydroxyphenyl)acetic acid (EDDHA) is a compound of the formula (XIII) and has the CAS no. 1170-02-1.

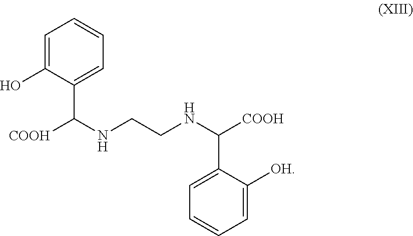 [SMILES: O=C(O)C(NCCNC(C(=O)O)c1ccccc1O)c1ccccc1O]

Diphosphoric acid forms during the association of two phosphoric acid molecules with the cleavage of water. Diphosphoric acid has the structure of the formula (XIV) and has the CAS no. 2466-09-3.

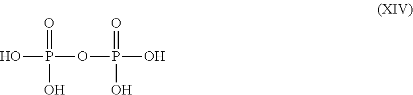

A suitable physiologically acceptable salt is, for example, the mono-, di-, tri-, or tetrasodium salt of the compound, or the mono-, di-, tri-, or tetrapotassium salt of this compound. The disodium salt of diphosphoric acid is also called disodium pyrophosphate.

Hydrated tin oxides in the context of the present invention are understood to be compounds which are tin dioxides (SnO2) with different amounts of bound water. Hydrated tin oxides are formed, for example, by the acidification of alkaline solutions of stannates, i.e., the salts of stannic acid H2[Sn(OH)6]. Hydrated tin oxides can be described by the general formula (SnO2)(H₂Ox). The physiologically acceptable salts of hydrated tin oxide are the physiologically acceptable stannates, such as, for example, potassium hexahydrostannate(IV) {K₂[Sn(OH)₆]}, sodium hexahydro stannate {Na₂[Sn(OH]₆]}, or also calcium hexahydrostannate {Ca[Sn(OH)₆]}.

It emerged during the work leading to this invention that the addition of one or more stabilizers (A3) from the previously described group to preparation (A) assures that preparation (A), which includes hydrogen peroxide (A2), can be packaged in the special sachet of the invention and stored, without the sachet, which has a barrier layer with a penetration barrier effect for gases and water vapor, swelling or bursting.

In this regard, very specific stabilizers have proven to be very particularly effective. The use of one or more stabilizers from the group comprising 2,6-dipicolinic acid, 1-hydroxyethane-1,1-diphosphonic acid (HEDP), diphosphoric acid, benzoic acid, and/or the physiologically acceptable salts thereof has emerged as being explicitly very particularly well suited for use in the sachet of the invention.

In another particularly preferred embodiment, a product of the invention is characterized in that preparation (A) includes one or more stabilizers (A3) from the group comprising 2,6-dipicolinic acid, 1-hydroxyethane-1,1-diphosphonic acid (HEDP), benzoic acid, diphosphoric acid, and/or the physiologically acceptable salts thereof.

In order to suppress the decomposition of hydrogen peroxide (A2), as occurs with gas formation, especially reliably and effectively, it is of very particular advantage to use two different, preferably three different, and even more preferably four different particularly suitable stabilizers in composition (A).

In another particularly preferred embodiment, a product of the invention is characterized in that preparation (A) includes

(A31) 2,6-dipicolinic acid and/or a physiologically acceptable salt thereof and
(A32) 1-hydroxyethane-1,1-diphosphonic acid (HEDP) and/or a physiologically acceptable salt thereof.

In another particularly preferred embodiment, a product of the invention is characterized in that preparation (A) includes

(A31) 2,6-dipicolinic acid and/or a physiologically acceptable salt thereof and
(A32) 1-hydroxyethane-1,1-diphosphonic acid (HEDP) and/or a physiologically acceptable salt thereof and
(A33) benzoic acid and/or a physiologically acceptable salt thereof.

In another particularly preferred embodiment, a product of the invention is characterized in that preparation (A) includes

(A31) 2,6-dipicolinic acid and/or a physiologically acceptable salt thereof and
(A32) 1-hydroxyethane-1,1-diphosphonic acid (HEDP) and/or a physiologically acceptable salt thereof and
(A34) diphosphoric acid and/or a physiologically acceptable salt thereof.

In another particularly preferred embodiment, a product of the invention is characterized in that preparation (A) includes

(A31) 2,6-dipicolinic acid and/or a physiologically acceptable salt thereof and
(A32) 1-hydroxyethane-1,1-diphosphonic acid (HEDP) and/or a physiologically acceptable salt thereof and
(A33) benzoic acid and/or a physiologically acceptable salt thereof and
(A34) diphosphoric acid and/or a physiologically acceptable salt thereof.

To further optimize the storage stability, the stabilizer(s) from group (A3) is/are preferably used in specific amount ranges in preparation (A). An increase in the storage stability could be observed even at low amounts employed of stabilizers (A3). The decomposition of hydrogen peroxide could be prevented to a satisfactory extent, however, if stabilizer(s) (A3) was/were added in a specific amount range to preparation (A). Therefore, stabilizer(s) (A3) is/are employed in preparation (A), based on the total weight of preparation (A), in particular in a total amount of 0.05 to 5.0% by weight, preferably of 0.15 to 2.5% by weight, more preferably 0.25 to 1.5% by weight, and particularly preferably 0.35 to 1.0% by weight in preparation (A). The calculation basis for these quantitative data in % by weight in this case is the total weight of all stabilizers from group (A3), which is related to the total weight of preparation (A).

In another particularly preferred embodiment, a product of the invention is characterized in that preparation (A) includes, based on the total weight of preparation (A), one or more stabilizers (A3) in a total amount of 0.05 to 5.0% by weight, preferably of 0.15 to 2.5% by weight, more preferably 0.25 to 1.5% by weight, and particularly preferably 0.35 to 1.0% by weight.

An explicitly very particularly preferred product of the invention is characterized in that preparation (A) includes, based on the total weight of preparation (A),

(A31) 0.05 to 2.5% by weight of 2,6-dipicolinic acid and/or a physiologically acceptable salt thereof and
(A32) 0.05 to 1.0% by weight of 1-hydroxyethane-1,1-diphosphonic acid (HEDP) and/or a physiologically acceptable salt thereof.

An explicitly very particularly preferred product of the invention is characterized further in that preparation (A) includes, based on the total weight of preparation (A),

(A33) 0.01 to 0.5% by weight of benzoic acid and/or a physiologically acceptable salt thereof and
(A34) 0.01 to 0.5% by weight of diphosphoric acid and/or a physiologically acceptable salt thereof.

An explicitly very particularly preferred product of the invention is characterized further in that preparation (A) includes, based on the total weight of preparation (A),

(A31) 0.05 to 2.5% by weight of 2,6-dipicolinic acid and/or a physiologically acceptable salt thereof and
(A32) 0.05 to 1.0% by weight of 1-hydroxyethane-1,1-diphosphonic acid (HEDP) and/or a physiologically acceptable salt thereof and
(A33) 0.01 to 0.5% by weight of benzoic acid and/or a physiologically acceptable salt thereof.

An explicitly very particularly preferred product of the invention is characterized further in that preparation (A) includes, based on the total weight of preparation (A),

(A31) 0.05 to 2.5% by weight of 2,6-dipicolinic acid and/or a physiologically acceptable salt thereof and
(A32) 0.05 to 1.0% by weight of 1-hydroxyethane-1,1-diphosphonic acid (HEDP) and/or a physiologically acceptable salt thereof and
(A34) 0.01 to 0.5% by weight of diphosphoric acid and/or a physiologically acceptable salt thereof.

An explicitly very particularly preferred product of the invention is characterized further in that preparation (A) includes, based on the total weight of preparation (A),

(A31) 0.05 to 1.0% by weight of 2,6-dipicolinic acid and/or a physiologically acceptable salt thereof and
(A32) 0.05 to 1.0% by weight of 1-hydroxyethane-1,1-diphosphonic acid (HEDP) and/or a physiologically acceptable salt thereof and
(A33) 0.01 to 0.5% by weight of benzoic acid and/or a physiologically acceptable salt thereof and
(A34) 0.01 to 0.5% by weight of diphosphoric acid and/or a physiologically acceptable salt thereof.

Preparation (A) located in sachet (S) includes the essential ingredients in an aqueous or aqueous-alcoholic carrier which can be, for example, a cream, an emulsion, a gel, or a surfactant-containing foaming solution as well. In order to adjust the desired properties of these delivery forms, preparation (A) can include furthermore additional active substances, auxiliary substances, and additives.

Preparation (A) can also include still other ingredients. Preparation (A) can contain, for example, in addition also one or more fatty components from the group comprising C₁₂-C₃₀ fatty alcohols, C₁₂-C₃₀ fatty acid triglycerides, C₁₂-C₃₀ fatty acid monoglycerides, C₁₂-C₃₀ fatty acid diglycerides, and/or hydrocarbons.

Preferably, in addition a surface-active substance can be added to preparation (A), wherein such surface-active substances depending on the field of application can be called surfactants or emulsifiers: they are preferably selected from anionic, zwitterionic, amphoteric, and nonionic surfactants and emulsifiers.

Agents suitable according to the invention are characterized in that the agent includes in addition at least one anionic surfactant. Preferred anionic surfactants are fatty acids, alkyl sulfates, alkyl ether sulfates, and ether carboxylic acids having 10 to 20 C atoms in the alkyl group and up to 16 glycol ether groups in the molecule.

Agents suitable according to the invention are characterized in that the agent includes in addition at least one zwitterionic surfactant. Preferred zwitterionic surfactants are betaines, N-alkyl-N,N-dimethylammonium glycinates, N-acylaminopropyl-N,N-dimethylammonium glycinates, and 2-alkyl-3-carboxymethyl-3-hydroxyethyl imidazolines. A preferred zwitterionic surfactant is known by the INCI name Cocamidopropyl Betaine.

Agents suitable according to the invention are characterized in that the agent includes in addition at least one amphoteric surfactant. Preferred amphoteric surfactants are N-alkylglycines, N-alkylpropionic acids, N-alkylaminobutyric acids, N-alkyliminodipropionic acids, N-hydroxyethyl-N-alkylamidopropylglycines, N-alkyltaurines, N-alkylsarco sines, 2-alkylaminopropionic acids, and alkylaminoacetic acids. Particularly preferred amphoteric surfactants are N-cocoalkyl aminopropionate, cocoacylaminoethylamino propionate, and C₁₂-C₁₈ acylsarcosine.

It has proven advantageous, furthermore, for the agents to include other non-ionogenic surface-active substances. Preferred nonionic surfactants are alkyl polyglycosides and alkylene oxide adducts to fatty alcohols and fatty acids with in each case 2 to 30 mol of ethylene oxide per mole of fatty alcohol or fatty acid. Preparations with excellent properties are likewise obtained if they include fatty acid esters of ethoxylated glycerol as the nonionic surfactants.

The nonionic, zwitterionic, or amphoteric surfactants are used in proportions of 0.1 to 45% by weight, preferably 1 to 30% by weight, and very especially preferably of 1 to 15% by weight, based on the total amount of the ready-to-use agents.

Preparation (A) can include in addition at least one thickener as well. There are no basic restrictions with regard to these thickeners. Both organic and purely inorganic thickeners may be used. Suitable thickeners are anionic, synthetic polymers, cationic, synthetic polymers, naturally occurring thickeners, such as nonionic guar gums, scleroglucan gums or xanthan gums, gum arabic, gum ghatti, karaya gum, tragacanth gum, carrageenan gum, agar-agar, locust bean gum, pectins, alginates, starch fractions, and derivatives such as amylose, amylopectin, and dextrins, as well as cellulose derivatives such as, for example, methylcellulose, carboxyalkylcelluloses, and hydroxyalkylcelluloses, nonionic, fully synthetic polymers, such as polyvinyl alcohol or polyvinylpyrrolidinone; as well as inorganic thickeners, in particular phyllosilicates such as, for example, bentonite, in particular smectites, such as montmorillonite or hectorite.

Further, preparation (A) can include other active substances, auxiliary substances, and additives, such as, for example, nonionic polymers such as, for example, vinylpyrrolidinone/vinyl acrylate copolymers, polyvinylpyrrolidinone, vinylpyrrolidinone/vinyl acetate copolymers, polyethylene glycols, and polysiloxanes; additional silicones such as volatile or nonvolatile, straight-chain, branched or cyclic, crosslinked or noncrosslinked polyalkylsiloxanes (such as dimethicones or cyclomethicones), polyarylsiloxanes, and/or polyalkylarylsiloxanes, particularly polysiloxanes with organofunctional groups, such as substituted or unsubstituted amines (amodimethicones), carboxy, alkoxy, and/or hydroxyl groups (dimethicone copolyols), linear polysiloxanes (A)-polyoxyalkylene (B) block copolymers, grafted silicone polymers; cationic polymers such as quaternized cellulose ethers, polysiloxanes with quaternary groups, dimethyldiallylammonium chloride polymers, acrylamide-dimethyldiallylammonium chloride copolymers, dimethylaminoethyl methacrylate-vinylpyrrolidinone copolymers quaternized with diethylsulfate, vinylpyrrolidinone-imidazolinium-methochloride copolymers, and quaternized polyvinyl alcohol; zwitterionic and amphoteric polymers; anionic polymers such as, for example, polyacrylic acids or crosslinked polyacrylic acids; structurants such as glucose, maleic acid, and lactic acid, hair-conditioning compounds such as phospholipids, for example, lecithin and kephalins; perfume oils, dimethyl isosorbide, and cyclodextrins; fiber structure-improving active substances, particularly mono-, di-, and oligosaccharides such as, for example, glucose, galactose, fructose, fruit sugar, and lactose; dyes for coloring the agent; antidandruff agents such as piroctone olamine, zinc omadine, and climbazole; amino acids and oligopeptides; protein hydrolysates with an animal and/or vegetable base, and in the form of their fatty acid condensation products or optionally anionically or cationically modified derivatives; fatty substances and vegetable oils; light stabilizers and UV blockers; active substances such as panthenol, pantothenic acid, pantolactone, allantoin, pyrrolidinone carboxylic acids, and salts thereof, as well as bisabolol; polyphenols, particularly hydroxycinnamic acids, 6,7-dihydroxycoumarins, hydroxybenzoic acids, catechins, tannins, leukoanthocyanidins, anthocyanidins, flavanones, flavones, and flavonols; ceramides or pseudoceramides; vitamins, provitamins, and vitamin precursors; plant extracts; fats and waxes such as fatty alcohols, beeswax, montan wax, and paraffins; swelling and penetration agents such as glycerol, propylene glycol monoethyl ether, carbonates, hydrogen carbonates, guanidines, ureas, and primary, secondary, and tertiary phosphates; opacifiers such as latex, styrene/PVP and styrene/acrylamide copolymers; pearlescent agents such as ethylene glycol mono- and distearate and PEG-3 distearate, as well as pigments.

The selection of these additional substances is made by the skilled artisan according to the desired properties of the agents. In regard to other facultative components and the employed amounts of said components, reference is made expressly to relevant handbooks known to the skilled artisan. The additional active and auxiliary substances are used in the agents of the invention preferably in each case in amounts of 0.0001 to 25% by weight, in particular of 0.0005 to 15% by weight, based in each case on the total weight of preparation (A).

Preparation (A) is packaged according to the invention in a sachet (S), wherein the wall of sachet (S) comprises

(S1) a first layer made of a first polymeric material and
(S2) a second layer made of a second polymeric material and
(SBa) a barrier layer, which has a penetration barrier effect for gases and water vapor.

In other words, preparation (A) is packaged in a sachet (S), wherein sachet (S) comprises a multilayer composite system, which has

(S1) a first layer made of a first polymeric material and
(S2) a second layer made of a second polymeric material and
(SBa) a barrier layer, which has a penetration barrier effect for gases and water vapor.

A sachet (S) in the context of the present invention is a small package in bag or pouch form. The capacity of the sachet can be, for example, 5 to 1000 mL, preferably 10 to 200 mL, and particularly preferably 20 to 50 mL.

Sachet (S) preferably comprises a multilayer composite system; i.e., in other words, the sachet is made of a composite film that comprises a plurality of layers. This multilayer film represents the wall or outer envelope of the sachet. As previously described, a sachet is generally produced by gluing, pressing, or heat sealing of two film pieces lying one on top of the other (the sachet being filled simultaneously with preparation (A)); i.e., a sachet is sealed on all edges. The sachet can be opened, for example, by tearing or cutting open.

The composite film used preferably for producing the sachet comprises at least three layers,

(S1) a first layer made of a first polymeric material and
(S2) a second layer made of a second polymeric material and
(SBa) a barrier layer, which has a penetration barrier effect for gases and water vapor.

The thickness of the composite film can be configured in this case such that there is a sufficient mechanical stability, but simultaneously the film, and thereby the sachet made of the film, is so flexible that complete removal of preparation (A) from the opened sachet (S) is possible by pressing together or pressing out of the opened film pouch. These requirements are fulfilled in particular if the multilayer composite system is a film comprising a plurality of layers with a total thickness of 21 μm (micrometers) to 2.0 mm (millimeters), preferably of 30 μm (micrometers) to 1.0 mm (millimeters), more preferably of 50 μm (micrometers) to 500 μm (micrometers), and very particularly preferably of 60 μm (micrometers) to 200 μm (micrometers).

An explicitly very particularly preferred product of the invention for this reason is characterized in that the wall of the sachet is a film comprising a plurality of layers with a total thickness of 21 μm (micrometers) to 2.0 mm (millimeters), preferably of 30 μm to 1.0 mm, more preferably of 50 μm to 500 μm, and very particularly preferably of 60 μm (micrometers) to 200 μm (micrometers).

The terms “multilayer composite system” and “multilayer composite film” are to be understood as synonymous in the context of the present invention. The total thickness of the film in the context of the present invention is understood to mean the sum of the thicknesses of all individual layers making up the film.

The multilayer composite system (i.e., the multilayer composite film) comprises at least three layers,

(S1) a first layer made of a first polymeric material and
(S2) a second layer made of a second polymeric material and
(SBa) a barrier layer, which has a penetration barrier effect for gases and water vapor.

The layers can also be called plies. All layers run parallel to the surface of the composite film.

The first polymeric material of the first layer (S1) according to the invention is an organic polymeric material. The second polymeric material of the second layer (S2) according to the invention is likewise an organic polymeric material.

The arrangement of the layers (S1), (S2), and (SBa) can be different in this case, the wall of the sachet can comprise further one or more additional layers, apart from the three layers (S1), (S2), and (SBa).

If the multilayer composite system (i.e., the multilayer composite film) comprises three layers, the following arrangements are possible, for example:

An arrangement of the invention is, when viewed from the inside toward the outside:

*interior*-layer (S1)-layer (S2)-barrier layer (SBa)-*outer side*
In this case, the multilayer film consists of three layers, wherein layer (S1) lies on the inside and is in contact with preparation (A). Layers (S1) and (S2) are adjacent to one another. So that the two layers can be differentiated, the polymeric materials constituting the two layers (S1) and (S2) are different. Barrier layer (SBa) is outermost. In films with this layering, for example, layer (S1) can function as a polymeric carrier layer to which the second polymers layer (S2) is applied. The side adjacent to (S2) (i.e., the outer side) is then provided with the barrier layer. The three layers (S1), (S2), and (SBa) together form a multilayer film in this way, whose total thickness is preferably 30 μm to 1.0 mm.

A further arrangement of the invention is, when viewed from the inside toward the outside:

*interior*-barrier layer (SBa)-layer (S1)-layer (S2)-*outer side*
In this case, the multilayer film consists of three layers, wherein the barrier layer (SBa) lies innermost and is in contact with preparation (A). Layers (S1) and (S2) are adjacent to one another. So that the two layers can be differentiated, the polymeric materials constituting the two layers (S1) and (S2) are different. Layer (S2) is outermost. The three layers (SBa), (S1), and (S2) together form a multilayer film in this way, whose total thickness is preferably 30 μm to 1.0 mm.

The most preferred layer arrangement occurs, if barrier layer (SBa) is arranged between the two polymer layers (S1) and (S2). In this arrangement as well, the multilayer composite system can also comprise one or more additional layers, apart from the three layers (S1), (S2), and (SBa).

A very particularly preferred product of the invention is furthermore characterized in that the barrier layer (SBa) is arranged between the two polymer layers (S1) and (S2).

In this very particularly preferred embodiment, the arrangement is, when viewed from the inside toward the outside:

*interior*-layer (S1)-barrier layer (SBa)-layer (S2)-*outer side*
In this case, the multilayer film consists of three layers, wherein layer (S1) is innermost and is in contact with preparation (A). Layer (S1) has contact with barrier layer (SBa), and barrier layer (SBa) in turn has contact with layer (S2). In this layer, layers (S1) and (S2) are not adjacent to one another but are separated by barrier layer (SBa). In this arrangement, layers (S1) and (S2) can consist in principle of the same polymeric material, but it is preferable, however, if the two layers (S1) and (S2) consist of different polymeric materials. The three layers (S1), (SBa), and (S2) together form a multilayer film in this way, whose total thickness is preferably 30 μm to 1.0 mm. The particular advantage of this arrangement is that the (often very thin) barrier layer (SBa) is located neither on the inner nor the outer surface of the multilayer film, but is protected in the direction of the inner side by polymer layer (S1) and in the direction of the outer side by polymer layer (S2). In this arrangement, mechanical abrasion or mechanical destruction of barrier layer (SBa) can be prevented in the best possible way.

The wall of sachet (S) of the invention comprises a first layer (S1) made of a first polymeric material. The first polymeric material can be a layer made of one polymer type or also a layer made of a polymer mixture. This first layer (S1) can function, for example, as a polymeric carrier material; i.e., in the production of the film, a layer or a film made of polymeric material (S1) can be provided and then sprayed, laminated, or coated with the other layers of the invention. Preferably first layer (S1) consists of polypropylene, polyethylene, polyester, polyamide, or polyvinyl alcohol. Very particularly preferably, the first layer (S1) consists of polypropylene.

A particularly preferred product of the invention is characterized in that the wall of sachet (S) comprises
(S1) a first layer made of a polymeric material, which is selected from the group comprising polypropylene, polyethylene, polyester, polyamide, and/or polyvinyl alcohol.

A very particularly preferred product of the invention is characterized in that the wall of sachet (S) comprises
(S1) a first layer made of polypropylene.

Polypropylene is also called alternatively poly(1-methylethylene) and is a thermoplastic polymer, which belongs to the group of polyolefins. Polypropylene is produced by polymerization of propylene (propene) with use of various catalysts. Thus, polypropylene can be produced, for example, by stereospecific polymerization of propylene in the gas phase or in suspension according to Giulio Natta. Polypropylenes of the invention can be isotactic and thereby highly crystalline, but also syndiotactic or amorphous. The control of the average relative molar mass can occur, for example, by setting a specific hydrogen partial pressure during the polymerization of propene. For example, polypropylene can have an average relative molar mass of about 150,000 to 1,500,000 g/mol. The processing of polypropylene can occur, for example, by extrusion and stretch blow molding, or by compression molding, calendering, thermoforming, and cold forming.

The thickness of first layer (S1) can be, for example, in the range of 20.0 to 300.0 μm (micrometers) and preferably constitutes a layer thickness of 40.0 to 200.0 μm (micrometers), more preferably of 50.0 to 100.0 μm (micrometers), and particularly preferably of 60.0 to 90.0 μm (micrometers).

A particularly preferred product of the invention is characterized in that the wall of sachet (S) comprises
(S1) a first layer made of the first polymeric material with a layer thickness of 20.0 to 300.0 μm (micrometers), preferably of 40.0 to 200.0 μm, more preferably of 50.0 to 100.0 μm, and particularly preferably of 60.0 to 90.0 μm (micrometers).

An explicitly very particularly preferred product of the invention is characterized in that the wall of sachet (S) comprises
(S1) a first layer made of polypropylene with a layer thickness of 20.0 to 300.0 μm (micrometers), preferably of 40.0 to 200.0 μm, more preferably of 50.0 to 100.0 μm, and particularly preferably of 60.0 to 90.0 μm (micrometers).

Furthermore, the multilayer film, from which the sachet is made, comprises a second layer (S2) made of a second polymeric material. The second polymeric material can be a layer made of one polymer type or also a layer made of a polymer mixture. During the production of the multilayer film, for example, the second layer (S2) can be sprayed, applied, or layered onto carrier layer (S1) either before or after the application of barrier layer (SBa). It is also conceivable, however, that second layer (S2) functions as a carrier layer, to which then barrier layer (SBa) and first polymers layer (S1) are applied.

Depending on the previously described sequence of the layering, the first polymeric material of first layer (S1) and the second polymeric material of second layer (S2) can be either the same (provided that both layers are not in contact with one another) or also different. Layers (S1) and (S2) are preferably fabricated of different polymeric materials (i.e., different polymers or polymer mixtures). Second layer (S2) can consist of polypropylene, polyethylene, polyester, and/or polyamide. Very particularly preferably, second layer (S2) consists of polyethylene terephthalate.

A particularly preferred product of the invention is characterized in that the wall of sachet (S) comprises
(S2) a second layer made of polyethylene terephthalate.

Polyethylene terephthalate (PET) is a polymer from the polyester group. Polyethylene terephthalate can be produced, for example, by the transesterification of dimethyl terephthalate with ethylene glycol at higher temperatures. Methanol, which is removed by distillation, is cleaved off in this transesterification reaction. The arising bis(2-hydroxyethyl)terephthalate is converted to PET by polycondensation, wherein ethylene glycol forms again. Another method for producing polyethylene terephthalate is the direct polycondensation of ethylene glycol and terephthalic acid at high temperatures with the removal of the forming water by distillation.

The thickness of first layer (S2) can be, for example, in the range of 1.0 to 100.0 μm (micrometers) and preferably constitutes a layer thickness of 2.5 to 50.0 μm, more preferably of 5.0 to 25.0 μm, and particularly preferably of 10.0 to 20.0 μm (micrometers).

A particularly preferred product of the invention is characterized in that the wall of sachet (S) comprises
(S2) a second layer made of a second polymeric material with a layer thickness of 1.0 to 100.0 μm (micrometers), preferably of 2.5 to 50.0 μm (micrometers), more preferably of 5.0 to 25.0 μm (micrometers), and particularly preferably of 10.0 to 20.0 μm (micrometers).

An explicitly very particularly preferred product of the invention is characterized in that the wall of sachet (S) comprises
(S2) a second layer made of polyethylene terephthalate with a layer thickness of 1.0 to 100.0 μm (micrometers), preferably of 2.5 to 50.0 μm (micrometers), more preferably of 5.0 to 25.0 μm (micrometers), and particularly preferably of 10.0 to 20.0 μm (micrometers).

As the third layer essential to the invention, the multilayer composite system (i.e., the multilayer composite film) of sachet (S) comprises a barrier layer (SBa), which has a penetration barrier effect for gases and water vapor.

Layers (S1) and (S2) of the composite film consist of organic polymeric materials. Organic polymers usually have an insufficient barrier effect against gases and water vapor. If aqueous preparation (A) is packaged in a sachet made of a multilayer film, comprising only the two organic polymer layers (S1) and (S2), water vapor can escape unimpeded, so that the water content in the composition changes in an unacceptable way upon longer storage.

In order to minimize selectively the uncontrolled escaping of water vapor out of the sachet, the organic polymer layers (S1) and (S2) are therefore used in the composite with a barrier layer (SBa).

Barrier layer (SBa) has a penetration barrier effect for gases and water vapor. This means according to the invention that barrier layer (SBa) reduces the permeation rate of water vapor and of gases through the film.

A film of the invention, which has a barrier layer (SBa) apart from layers (S1) and (S2), therefore has a reduced water vapor permeability and a reduced gas permeability compared with a comparable film (with same total thickness), which only has the two layers (S1) and (S2) but no barrier layer (SBa).

Barrier layer (SBa) is, for example, a thin layer, which comprises an inorganic material, wherein the inorganic material can be applied with the aid of vacuum coating techniques (e.g., PVD “physical vapor deposition” or CVD “chemical vapor deposition”) to the organic polymer layer (S1) and/or (S2).

Barrier layer (SBa) is a layer, which comprises at least one inorganic material, so that, for example, aluminum, aluminum oxides, magnesium, magnesium oxides, silicon, silicon oxides, titanium, titanium oxides, tin, tin oxides, zirconium, zirconium oxides, and/or carbon may be used for this.

The production of films with barrier layers made of inorganic material is described, for example, in the publication EP 1036813 A1, which is incorporated herein in its entirety by reference.

Oxides, which can be selected from the group comprising aluminum oxides, magnesium oxides, silicon oxides, titanium oxides, tin oxides, and/or zirconium oxides, are used particularly preferably in this regard. Barrier layer (SBa) made of an inorganic material is located with very particular preference between the two polymer layers (S1) and (S2).

A particularly preferred product of the invention is characterized in that the wall of sachet (S) has
(SBa) a barrier layer that lies between the two polymer layers (S1) and (S2) and includes aluminum oxides, magnesium oxides, silicon oxides, titanium oxides, tin oxides, zirconium oxides, or mixtures thereof.

Within the group of metal oxides, silicon dioxide is very especially well-suited to prevent the evaporation of water from preparation (A) through sachet (S).

A very particularly preferred product of the invention is characterized in that the wall of sachet (S) has
(SBa) a barrier layer that lies between the two polymer layers (S1) and (S2) and includes silicon oxides.

Barrier layer (SBa) can also comprise a thin layer of inorganic-organic hybrid polymers; these polymers are known in the literature under the technical term ORMOCER polymers. A typical ORMOCER polymer can be prepared, for example, by hydrolytic polycondensation of an organofunctional silane, e.g., with an aluminum compound and optionally with an inorganic oxide component. Corresponding syntheses are disclosed, for example, in the publication EP 0792846 B1, which is incorporated herein in its entirety by reference. Inorganic-organic hybrid polymers (ORMOCER polymers) have both inorganic and organic network structures.

The inorganic silicate network structure can be built up in the sol-gel process by the controlled hydrolysis and condensation of alkoxysilanes. Because additional metal alkoxides are included in the sol-gel process, the silicate network can be selectively modified. An organic network is built in addition by polymerization of organofunctional groups, which are incorporated into the material by organoalkoxysilanes. The ORMOCER polymers prepared in this way can be applied to layers (S1) and/or (S2), for example, by conventional application techniques (spraying, brushing, etc.).

A particularly preferred product of the invention is characterized in that the wall of sachet (S) has
(SBa) a barrier layer that lies between the two polymer layers (S1) and (S2) and includes one or more inorganic-organic hybrid polymers (ORMOCER polymers).

Furthermore, it is also possible that the multilayer composite system (i.e., the multilayer composite film), which represents the wall of the sachet, has a barrier layer (SBa), which comprises both inorganic oxide components and inorganic-organic hybrid polymers (ORMOCER polymers). In addition, barrier layer (SBa) can also comprise another organic polymeric material, which itself has no barrier effect, but increases, for example, the mechanical stability of the barrier layer, simplifies production, or brings about a better gluing of layers (SBa) and (S1) and/or (S2).

A particularly preferred product of the invention is characterized in that the wall of sachet (S) has
(SBa) a barrier layer that lies between the two polymer layers (S1) and (S2) and includes aluminum oxides, magnesium oxides, silicon oxides, titanium oxides, tin oxides, zirconium oxides, or mixtures thereof, and includes furthermore one or more inorganic-organic hybrid polymers (ORMOCER polymers).

The thicker the barrier layer (SBa), the greater or stronger the penetration barrier effect for gases and water vapor. The thickness of barrier layer (SBa) can therefore be selected as a function of the desired barrier layer effect. Barrier layer (SBa) can have, for example, a layer thickness of 1 to 1000 nm (nanometers). Barrier layer (SBa) preferably has a layer thickness of 5 to 500 nm, more preferably of 10 to 250 nm, and particularly preferably of 10 to 150 nm (nanometers).

A particularly preferred product of the invention is characterized in that the wall of sachet (S) has
(SBa) a barrier layer that lies between the two polymer layers (S1) and (S2) and has a layer thickness of 1 to 1000 nm (nanometers), preferably of 5 to 500 nm (nanometers), more preferably of 10 to 250 nm (nanometers), and particularly preferably of 10 to 150 nm (nanometers).

Apart from the layers described thus far (S1), (S2), and (SBa), the multilayer composite system of the invention (i.e., the multilayer composite film) can comprise in addition also one or more other layers. These additional layers can be, for example, intermediate layers (SZ) and/or adhesive layers (SK1).

For example, the films can have other intermediate layers (SZ) in order to increase the mechanical stability. Intermediate layers can also prevent or minimize the permeation of polymers or remaining monomers from a polymer layer in preparation (A).

In order to increase the bond strength, the films can comprise in addition also one or more adhesive layers. Even if there is concern about delamination (i.e., a detaching or the formation of an air space) between two layers, a further adhesive layer can also be used in addition.

A particularly preferred product of the invention is characterized in that the wall of sachet (S) includes, apart from the first layer made of the first polymeric material (S1), the second layer made of the second polymeric material (S2), and the barrier layer (SBa), in addition one or more further layers, which are selected from
intermediate layers (SZ) and/or
adhesive layers (SK).

If sachet (S) of the invention includes still further layers in addition to layers (S1), (S2), and (SBa), layer arrangements that are suitable and according to the invention are described hereinafter.

An arrangement of the invention is, when viewed from the inside toward the outside:

*interior*-layer (S1)-first adhesive layer (SK1)-layer (S2)-second adhesive layer (SK2)-barrier layer (SBa)-*outer side*
*interior*-layer (S1)-adhesive layer (SK1)-layer (S2)-barrier layer (SBa)-*outer side*
*interior*-layer (S1)-layer (S2)-second adhesive layer (SK2)-barrier layer (SBa)-*outer side*
*interior*-barrier layer (SBa)-first adhesive layer (SK1)-layer (S1)-second adhesive layer (SK2)-layer (S2)-*outer side*
*interior*-barrier layer (SBa)-adhesive layer (SK)-layer (S1)-layer (S2)-*outer side*
*interior*-barrier layer (SBa)-layer (S1)-adhesive layer (SK)-layer (S2)-*outer side*
*interior*-layer (S1)-first adhesive layer (SK1)-barrier layer (SBa)-second adhesive layer (SK2)-layer (S2)-*outer side*
*interior*-layer (S1)-adhesive layer (SK)-barrier layer (SBa)-layer (S2)-*outer side*
*interior*-layer (S1)-barrier layer (SBa)-adhesive layer (SK)-layer (S2)-*outer side*

The product of the invention is used for the purpose of oxidative color changing. To this end, preparation (A), which is packaged in the sachet and which is the oxidizing agent preparation, is mixed with at least another preparation (B) to prepare the ready-to-use color change agent. To prevent incompatibilities or to prevent a premature reaction, preparations (A) and (B) are packaged separately from one another.

A particularly preferred product of the invention is characterized in that it comprises a preparation (B), packaged separately from preparation (A), wherein
preparation (B) includes at least one oxidation dye precursor and/or at least one alkalizing agent.

If an oxidative coloring is desired, preparation (B) includes at least one oxidation dye precursor. Oxidation dye precursors can be divided into developers and couplers, wherein the developers because of their higher sensitivity to oxygen are used mostly in the form of their physiologically acceptable salts (e.g., in the form of their hydrochlorides, hydrobromides, hydrogen sulfates, or sulfates). Coupler components during oxidative dyeing alone cause no significant coloring, but always require the presence of developer components.

The agents of the invention can also be used, furthermore, together with oxidation dyes. Oxidation dyes of this type include in addition at least one oxidation dye precursor, preferably at least one oxidation dye precursor of the developer type and at least one oxidation dye precursor of the coupler type. Particularly suitable oxidation dye precursors of the developer type are selected in this case from at least one compound from the group formed by p-phenylenediamine, p-toluylenediamine, 2-(2-hydroxyethyl)-p-phenylenediamine, 2-(1,2-dihydroxyethyl)-p-phenylenediamine, N,N-bis(2-hydroxyethyl)-p-phenylenediamine, 2-methoxymethyl-p-phenylenediamine, N-(4-amino-3-methylphenyl)-N-[3-(1H-imidazol-1-yl)propyl]amine, N,N′-bis(2-hydroxyethyl)-N,N′-bis(4-aminophenyl)-1,3-diaminopropan-2-ol, bis(2-hydroxy-5-aminophenyl)methane, 1,3-bis(2,5-diaminophenoxy)propan-2-ol, N,N′-bis(4-aminophenyl)-1,4-diazacyclo-heptane, 1,10-bis(2,5-diaminophenyl)-1,4,7,10-tetraoxadecane, p-aminophenol, 4-amino-3-methylphenol, 4-amino-2-aminomethylphenol, 4-amino-2-(1,2-dihydroxyethyl)phenol and 4-amino-2-(diethylaminomethyl)phenol, 4,5-diamino-1-(2-hydroxyethyl)pyrazole, 2,4,5,6-tetraaminopyrimidine, 4-hydroxy-2,5,6-triaminopyrimidine, 2-hydroxy-4,5,6-triaminopyrimidine, 2,3-diamino-6,7-dihydro-1H,5H-pyrazolo[ 1,2-a]pyrazol-1-one, and the physiologically acceptable salts thereof.

Especially suitable oxidation dye precursors of the coupler type in this case are selected from the group, formed by 3-aminophenol, 5-amino-2-methylphenol, 3-amino-2-chloro-6-methylphenol, 2-hydroxy-4-aminophenoxyethanol, 5-amino-4-chloro-2-methylphenol, 5-(2-hydroxyethyl)amino-2-methylphenol, 2,4-dichloro-3-aminophenol, 2-aminophenol, 3-phenylenediamine, 2-(2,4-diaminophenoxy)ethanol, 1,3-bis(2,4-diaminophenoxy)propane, 1-methoxy-2-amino-4-(2-hydroxyethylamino)benzene, 1,3-bis(2,4-diaminophenyl)propane, 2,6-bis(2′-hydroxyethylamino)-1-methylbenzene, 2-({3-[(2-hydroxyethyl)amino]-4-methoxy-5-methylphenyl}amino)ethanol, 2-({3-[(2-hydroxyethyl)amino]-2-methoxy-5-methylphenyl}amino)ethanol, 2-({3-[(2-hydroxyethyl)amino]-4,5-dimethylphenyl}amino)ethanol, 2-[3-morpholin-4-ylphenyl)amino]ethanol, 3-amino-4-(2-methoxyethoxy)-5-methylphenylamine, 1-amino-3-bis(2-hydroxyethyl)aminobenzene, resorcinol, 2-methylresorcinol, 4-chlororesorcinol, 1,2,4-trihydroxybenzene, 2-amino-3-hydroxypyridine, 3-amino-2-methylamino-6-methoxypyridine, 2,6-dihydroxy-3,4-dimethylpyridine, 3,5-diamino-2,6-dimethoxypyridine, 1-phenyl-3-methylpyrazol-5-one, 1-naphthol, 1,5-dihydroxy-naphthalene, 2,7-dihydroxynaphthalene, 1,7-dihydroxynaphthalene, 1,8-dihydroxynaphthalene, 4-hydroxyindole, 6-hydroxyindole, 7-hydroxyindole, 4-hydroxyindoline, 6-hydroxyindoline, 7-hydroxyindoline, or mixtures of said compounds or the physiologically acceptable salts thereof.

In addition, preparation (B) can also include one or more direct dyes. Suitable nonionic direct dyes can be selected from the group comprising HC Yellow 2, HC Yellow 4, HC Yellow 5, HC Yellow 6, HC Yellow 12, HC Orange 1, Disperse Orange 3, HC Red 1, HC Red 3, HC Red 7, HC Red 10, HC Red 11, HC Red 13, HC Red BN, HC Blue 2, HC Blue 11, HC Blue 12, Disperse Blue 3, HC Violet 1, Disperse Violet 1, Disperse Violet 4, Disperse Black 9, 1,4-diamino-2-nitrobenzene, 2-amino-4-nitrophenol, 1,4-bis(2-hydroxyethyl)amino-2-nitrobenzene, 3-nitro-4-(2-hydroxyethyl)aminophenol, 2-(2-hydroxyethyl)amino-4,6-dinitrophenol, 4-[(2-hydroxyethyl)amino]-3-nitro-1-methylbenzene, 1-amino-4-(2-hydroxyethyl)amino-5-chloro-2-nitrobenzene, 4-amino-3-nitrophenol, 1-(2′-ureidoethyl)amino-4-nitrobenzene, 2-[(4-amino-2-nitrophenyl)amino]benzoic acid, 4-[(3-hydroxypropyl)amino]-3-nitrophenol, 4-nitro-o-phenylenediamine, 6-nitro-1,2,3,4-tetrahydroquinoxaline, 2-hydroxy-1,4-naphthoquinone, picramic acid and salts thereof, 2-amino-6-chloro-4-nitrophenol, 4-ethylamino-3-nitrobenzoic acid, and 2-chloro-6-ethylamino-4-nitrophenol.

Suitable anionic direct dyes can be selected from the group comprising Acid Yellow 1, Yellow 10, Acid Yellow 23, Acid Yellow 36, Acid Orange 7, Acid Red 33, Acid Red 52, pigment Red 57:1, Acid blood 7, Acid Green 50, Acid Violet 43, Acid Black 1, Acid Black 52, bromophenol blue, and tetrabromophenol blue.

Suitable cationic direct dyes are cationic triphenylmethane dyes, such as, for example, Basic Blue 7, Basic Blue 26, Basic Violet 2, and Basic Violet 14, aromatic systems, substituted with a quaternary nitrogen group, such as, for example, Basic Yellow 57, Basic Red 76, Basic Blue 99, Basic Brown 16, and Basic Brown 17, cationic anthraquinone dyes, such as HC Blue 16 (Bluequat B), and direct dyes, containing a heterocycle that has at least one quaternary nitrogen atom, particularly Basic Yellow 87, Basic Orange 31, and Basic Red 51. The cationic direct dyes, which are marketed under the trademark Arianor, are also suitable cationic direct dyes according to the invention.

Coloring processes on keratin fibers typically take place in an alkaline environment. To treat keratin fibers and the skin as well as gently as possible, setting a too high pH is not desirable, however. It is preferred, therefore, if the pH of the ready-to-use agent is between 7 and 11, in particular between 8 and 10.5. pH values in the context of the present invention are pH values measured at a temperature of 22° C.

Preparation (B) can include at least one alkalizing agent. The alkalinizing agents that can be used to adjust the preferred pH according to the invention can be selected from the group formed by ammonia, alkanolamines, basic amino acids, and inorganic alkalinizing agents such as alkali (alkaline earth) metal hydroxides, alkali (alkaline earth) metal metasilicates, alkali (alkaline earth) metal phosphates, and alkali (alkaline earth) metal hydrogen phosphates. Preferred inorganic alkalinizing agents are magnesium carbonate, sodium hydroxide, potassium hydroxide, sodium silicate, and sodium metasilicate. Organic alkalinizing agents that can be used according to the invention are preferably selected from monoethanolamine, 2-amino-2-methylpropanol, and triethanolamine. The basic amino acids that can be used as alkalinizing agents of the invention are preferably selected from the group formed by arginine, lysine, ornithine, and histidine, especially preferably arginine. It has emerged in the context of studies for the present invention, however, that, furthermore, agents preferred according to the invention are characterized in that they include in addition an organic alkalinizing agent. An embodiment of the first subject of the invention is characterized in that the agent includes in addition at least one alkalinizing agents, which is selected from the group formed by ammonia, alkanolamines, and basic amino acids, particularly by ammonia, monoethanolamine, and arginine, or the acceptable salts thereof.

Preparation (B) can contain, furthermore, additional active substances, auxiliary substances, and additives, as they were already disclosed in the description of preparation (A).

### EXAMPLES

### Example 1

A 100-nm-thick layer of silicon dioxide SiOx was vapor deposited onto a film layer made of polyethylene terephthalate with a thickness of 12 μm (micrometers). Next, the SiOx layer was overcoated with about 3 g/m² of ORMOCER polymer and cured. A 70-μm (micrometers) thick layer of polypropylene was then applied to the ORMOCER layer. Sachets were produced from the film.

The sachets were filled with the following preparations (A) (all quantities are given in % by weight)

| | Example 1 | |
|---|---|---|
| | (according to | Example 2 |
| Ingredients | the invention) | (comparison) |
| Propylene glycol | 1.0 | 1.0 |
| Ceteareth-20 | 1.0 | 1.0 |
| Cetearyl alcohol | 3.4 | 3.4 |
| Steartrimonium chloride | 0.31 | 0.31 |
| Liquid paraffin | 0.3 | 0.3 |
| Etidronic acid (1- | 0.15 | — |
| hydroxyethane-1,1- | | |
| diphosphonic acid) | | |
| Pyridine-2,6-dicarboxylic acid | 0.10 | — |
| Disodium pyrophosphate | 0.10 | — |
| Sodium benzoate | 0.04 | — |
| (sodium salt of benzoic acid) | | |
| Potassium hydroxide | 0.1 | 0.1 |
| Isopropanol | 0.08 | 0.08 |
| Hydrogen peroxide | 12.0 | 12.0 |
| Water | to 100 | to 100 |
| | Example 3 | |
| | (according to | Example 4 |
| Ingredients | the invention) | (comparison) |
| Propylene glycol | 1.0 | 1.0 |
| Ceteareth-20 | 1.0 | 1.0 |
| Cetearyl alcohol | 3.4 | 3.4 |
| Steartrimonium chloride | 0.31 | 0.31 |
| Liquid paraffin | 0.3 | 0.3 |
| Etidronic acid (1- | 0.15 | — |
| hydroxyethane-1,1- | | |
| diphosphonic acid) | | |
| Pyridine-2,6-dicarboxylic acid | 0.10 | — |
| Disodium pyrophosphate | 0.10 | — |
| Sodium benzoate | 0.04 | — |
| (sodium salt of benzoic acid) | | |
| Potassium hydroxide | 0.1 | 0.1 |
| Isopropanol | 0.08 | 0.08 |
| Hydrogen peroxide | 6.0 | 6.0 |
| Water | to 100 | to 100 |

Each preparation in Examples 1 to 4 was filled into one of the previously described sachets. The sachets were then stored for 24 weeks at 40° C.

| | Example 1 | Example 2 | Example 3 | Example 4 |
|---|---|---|---|---|
| Storage for | Sachet not | Sachet burst | Sachet not | Sachet burst |
| 24 weeks, | swollen | | swollen | |
| 40° C. | | | | |
| Water loss | 3.51% | Not | 3.53% | Not |
| | acceptable | determinable | acceptable | determinable |

### Further Formulation Examples

A 100-nm-thick layer of silicon dioxide SiOx was vapor deposited onto a film layer made of polyethylene terephthalate with a thickness of 12 μm (micrometers). Next, the SiOx-layer was overcoated with about 3 g/m² of ORMOCER polymer and cured. A 70-μm (micrometers) thick layer of polypropylene was then applied to the ORMOCER layer. Sachets were produced from the film.

The sachets were filled with the following preparations (A) (all quantities are given in % by weight)

| Ingredients | Example 5 | Example 6 | Example 7 |
|---|---|---|---|
| Propylene glycol | 0.5 | 0.5 | 0.5 |
| Ceteareth-20 | 1.2 | 1.2 | 1.2 |
| Cetearyl alcohol | 3.6 | 3.6 | 3.6 |
| Liquid paraffin | 2.1 | 2.1 | 2.1 |
| Etidronic acid (1- | 0.25 | 0.25 | 0.25 |
| hydroxyethane-1,1- | | | |
| diphosphonic acid) | | | |
| Pyridine-2,6-dicarboxylic | 0.10 | 0.10 | 0.10 |
| acid | | | |
| Disodium pyrophosphate | 0.10 | 0.10 | 0.10 |
| Sodium benzoate | 0.05 | 0.05 | 0.05 |
| (sodium salt of benzoic acid) | | | |
| Potassium hydroxide | 0.15 | 0.15 | 0.15 |
| Hydrogen peroxide | 6.0 | 9.0 | 12.0 |
| Water | to 100 | to 100 | to 100 |

Each preparation in Examples 5 to 7 was filled into one of the previously described sachets.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A cosmetic product for the oxidative color changing of keratinic fibers, comprising
a preparation (A), which is packaged in a sachet (S), wherein
preparation (A) includes
(A1) water and
(A2) hydrogen peroxide and
(A3) at least one stabilizer from the group comprising 2,6-dipicolinic acid, 1-hydroxyethane-1,1-diphosphonic acid (HEDP), benzoic acid, salicylic acid, ethylenediaminetetraacetic acid (EDTA), ethylenediamine tetra(methylenephosphonic acid) (EDTMP), diethylenetriamine penta(methylenephosphonic acid) (DTPMP), amino tris(methylene-phosphonic acid) (ATMP), N,N-bis[2-[bis(carboxymethyl)amino]ethyl]glycine, ethylenediamine-N,N′-disuccinic acid (EDDS), 2-hydroxypropylenediamine-N,N′-disuccinic acid (HPDDS), ethylenediamine-N,N′-diglutaric acid (EDDG), ethylene-diamine-N,N′-bis(orthohydroxyphenyl)acetic acid (EDDHA), diphosphoric acid, hydrated tin oxide, and/or the physiologically acceptable salts thereof, and
the wall of sachet (S) comprises:
(S1) a first layer made of a first polymeric material and
(S2) a second layer made of a second polymeric material and
(SBa) a barrier layer, which has a penetration barrier effect for gases and water vapor.

2. The product according to claim 1, wherein preparation (A) comprises, based on the total weight of preparation (A), water (A1) in an amount of 60.0 to 97.0% by weight.

3. The product according to claim 1, wherein preparation (A) comprises, based on the total weight of preparation (A), hydrogen peroxide (A2) in an amount of 1.5 to 12.0% by weight.

4. The product according to claim 1, wherein preparation (A) wherein the one or more stabilizers (A3) is selected from the group consisting of 2,6-dipicolinic acid, 1-hydroxyethane-1,1-diphosphonic acid (HEDP), benzoic acid, diphosphoric acid, and the physiologically acceptable salts thereof.

5. The product according to claim 1, wherein preparation (A) includes, based on the total weight of preparation (A), the one or more stabilizers (A3) in a total amount of 0.05 to 5.0% by weight

6. The product according to claim 1, wherein preparation (A) includes, based on the total weight of preparation (A), the one or more stabilizers (A3) in a total amount of 0.25 to 1.5% by weight.

7. The product according to claim 1, wherein preparation (A) includes, based on the total weight of preparation (A), 0.05 to 2.5% by weight of 2,6-dipicolinic acid and/or a physiologically acceptable salt thereof, and 0.05 to 1.0% by weight of 1-hydroxyethane-1,1-diphosphonic acid (HEDP) and/or a physiologically acceptable salt thereof.

8. The product according to claim 1, wherein (A) includes, based on the total weight of preparation (A), 0.01 to 0.5% by weight of benzoic acid and/or a physiologically acceptable salt thereof, and 0.01 to 0.5% by weight of diphosphoric acid and/or a physiologically acceptable salt thereof.

9. The product according to claim 1, wherein the wall of the sachet is a film comprising a plurality of layers with a total thickness of 21 μm (micrometers) to 2.0 mm (millimeters).

10. The product according to claim 1, wherein the barrier layer (SBa) is arranged between the two polymer layers (S1) and (S2).

11. The product according to claim 1, wherein the first layer of the wall of sachet (S) comprises a polymeric material selected from the group consisting of polypropylene, polyethylene, polyester, polyamide, and polyvinyl alcohol.

12. The product according of claim 1, wherein the first layer of the wall of sachet (S) has a thickness of 20.0 to 300.0 μm (micrometers).

13. The product according to claim 1, wherein the second layer of the wall of sachet (S) includes polyethylene terephthalate.

14. The product according to claim 1, wherein the second layer of the wall of sachet (S) has a thickness of 1.0 to 100.0 μm (micrometers).

15. The product according to claim 1, wherein the barrier layer that lies between the two polymer layers in the wall of sachet (S) includes aluminum oxides, magnesium oxides, silicon oxides, titanium oxides, tin oxides, zirconium oxides, or mixtures thereof.

16. The product according to claim 1, wherein the barrier layer that lies between the two polymer layers in the wall of sachet (S) includes one or more inorganic-organic hybrid polymers (ORMOCER polymers).

17. The product according to claim 1, wherein the barrier layer that lies between the two polymer layers in the wall of sachet (S) has a layer thickness of 1 to 1000 nm (nanometers).

18. The product according to claim 1, wherein the barrier layer that lies between the two polymer layers in the wall of sachet (S) has a layer thickness 10 to 250 nm (nanometers).

19. The product according to claim 1, wherein the wall of sachet (S) further comprises, one or more further layers, which are selected from intermediate layers (SZ) and/or adhesive layers (SK).

20. The product according to claim 1, further comprising a preparation (B), packaged separately from preparation (A), wherein preparation (B) includes at least one oxidation dye precursor and/or at least one alkalizing agent.
